(19) United States
(12) Patent Application Publication
(10) Pub. No.: US 2018/0303451 A1
(43) Pub. Date: Oct. 25, 2018

(54) RADIATION IRRADIATION DETECTION SYSTEM, RADIATION GENERATION APPARATUS, AND RADIATION DETECTION APPARATUS

(71) Applicant: FUJIFILM CORPORATION, Tokyo (JP)

(72) Inventor: Kenji NAKAMURA, Kanagawa (JP)

(21) Appl. No.: 15/949,093

(22) Filed: Apr. 10, 2018

(30) Foreign Application Priority Data
Apr. 25, 2017 (JP) 2017-086708

(51) Int. Cl. A61B 6/00; G01T 1/208

(52) U.S. Cl. CPC A61B 6/542; G01T 1/208

(57) ABSTRACT

A radiation irradiation detection system includes a radiation generation apparatus that includes a radiation generation unit, and an emission control unit, and a radiation detection apparatus that includes a radiation detector, in which the radiation detection apparatus further includes a first wireless communication unit that outputs an emission permitting signal for permitting the radiation to be emitted as a wireless signal, in which the radiation generation apparatus further includes a second wireless communication unit that receives the emission permitting signal, and, in which, in a case where the emission permitting signal is received, the emission control unit causes the pulsed radiation to be emitted, and controls emission of the pulsed radiation by using the emission permitting signal received by the second wireless communication unit in a period in which the radiation is not emitted during an emission period of the pulsed radiation.

### CROSS REFERENCE TO RELATED APPLICATIONS

The present application claims priority under 35 U.S.C. § 119 to Japanese Patent Application No. 2017-086708 filed on Apr. 25, 2017. The above application is hereby expressly incorporated by reference, in its entirety, into the present application.

### BACKGROUND

### Technical Field

The present invention relates to a radiation irradiation detection system which emits radiation toward a subject and detects radiation transmitted through the subject, and a radiation generation apparatus and a radiation detection apparatus in the radiation irradiation detection system.

### Related Art

In the related art, there has been proposed a radiation irradiation detection system configured to include a radiation generation apparatus provided with an X-ray tube and the like, and a radiation detection apparatus provided with a radiation detector such as a flat panel detector (FPD) detecting radiation which is output from the radiation generation apparatus and is transmitted through a patient.

In such a radiation irradiation detection system, a preparation operation of the radiation detector is controlled by periodically resetting electric charge signals accumulated by leaking currents while radiation is not applied. During starting of radiation irradiation, preparation operation control transitions to electric charge accumulation control. In the electric charge accumulation control, an electric charge signal corresponding to a dose of radiation transmitted through a patient is accumulated in each pixel of a radiation image detector.

In order to cause the radiation detector to transition from the preparation operation control to the electric charge accumulation control according to the starting of radiation irradiation as mentioned above, the radiation generation apparatus and the radiation detection apparatus are required to be synchronized with each other.

As a method of synchronization between the radiation generation apparatus and the radiation detection apparatus, for example, a method is proposed in which a signal for permitting radiation to be emitted is transmitted from the radiation detection apparatus to the radiation generation apparatus in a wired or wireless manner, the radiation detection apparatus starts to emit radiation in a case where the emission permitting signal is received, and the radiation detector starts an electric charge accumulation operation.

For example, JP2014-57831A proposes that, in a radiation generation apparatus having an exposure switch which receives radiation irradiation instruction, in a case where a logical product of an irradiation instruction from the exposure switch and the emission permitting signal is true, radiation is emitted from the radiation generation apparatus.

However, for example, in a case where an emission permitting signal is transmitted from a radiation detection apparatus to a radiation generation apparatus as a wireless communication signal, the emission permitting signal may not be normally received by the radiation generation apparatus due to, for example, the influence of noise.

In this case, as described above, in a case where emission of radiation is controlled on the basis of a logical product of the emission permitting signal output from the radiation detection apparatus and the irradiation instruction from the exposure switch, the emission permitting signal cannot be normally received, and thus emission of radiation is stopped. In other words, capturing of a radiation image is wrongly stopped although the radiation image can be normally captured. Therefore, reimaging is required to be performed, and thus there is a problem in that a radiation exposure dose of a patient increases.

### SUMMARY

The present invention has been made in light of the problem, and an object thereof is to provide a radiation irradiation detection system, a radiation generation apparatus, and a radiation detection apparatus capable of reducing a radiation exposure dose of a patient without performing unnecessary reimaging.

According to the present invention, there is provided a radiation irradiation detection system including a radiation generation apparatus that includes a radiation generation unit generating radiation, and an emission control unit controlling emission of the radiation; and a radiation detection apparatus that includes a radiation detector detecting the radiation transmitted through a subject, in which the radiation detection apparatus further includes a first wireless communication unit that outputs an emission permitting signal for permitting the radiation to be emitted as a wireless signal, in which the radiation generation apparatus further includes a second wireless communication unit that receives the emission permitting signal, and, in which, in a case where the emission permitting signal is received, the emission control unit causes the pulsed radiation to be emitted, and controls emission of the pulsed radiation by using the emission permitting signal received by the second wireless communication unit in a period in which the radiation is not emitted during an emission period of the pulsed radiation.

In the radiation irradiation detection system of the present invention, the first wireless communication unit may output the emission permitting signal only in the period in which the radiation is not emitted.

In the radiation irradiation detection system of the present invention, the first wireless communication unit may continuously output the emission permitting signal, and the emission control unit may control emission of the pulsed radiation by using the emission permitting signal received in the period in which the radiation is not emitted among the emission permitting signals received by the second wireless communication unit.

In the radiation irradiation detection system of the present invention, in a case of stopping emission of the radiation, the first wireless communication unit may output an emission stopping signal for instructing emission of the radiation to be stopped as a wireless signal, and preferably continuously outputs the emission stopping signal during at least one cycle of the pulsed radiation.

In the radiation irradiation detection system of the present invention, in a case of stopping emission of the radiation, the first wireless communication unit may stop outputting of the emission permitting signal during at least one cycle of the pulsed radiation.

In the radiation irradiation detection system of the present invention, the radiation detection apparatus may output the emission permitting signal, and then may start an operation of accumulating a detection signal of the radiation in the radiation detector.

In the radiation irradiation detection system of the present invention, in a case where a preparation operation of the radiation detector is finished, the radiation detection apparatus may output the emission permitting signal, and may start an operation of accumulating a detection signal of the radiation in the radiation detector.

In the radiation irradiation detection system of the present invention, the radiation generation apparatus is preferably portable.

According to the present invention, there is provided a radiation generation apparatus including a radiation generation unit that generates radiation; an emission control unit that controls emission of the radiation; and a wireless communication unit that performs wireless communication, in which, in a case where an emission permitting signal as a wireless signal output from a radiation detection apparatus detecting the radiation transmitted through a subject is received by the wireless communication unit, the emission control unit causes the pulsed radiation to be emitted, and controls emission of the pulsed radiation by using the emission permitting signal received in a period in which the radiation is not emitted during an emission period of the pulsed radiation.

The radiation generation apparatus of the present invention is preferably portable.

According to the present invention, there is provided a radiation detection apparatus including a radiation detector that detects radiation transmitted through a subject; and a wireless communication unit that performs wireless communication, in which the wireless communication unit outputs an emission permitting signal for permitting the radiation to be emitted as a wireless signal to a radiation generation apparatus, and outputs the emission permitting signal in only a period in which the radiation is not emitted during an emission period of pulsed radiation emitted from the radiation generation apparatus in response to the emission permitting signal.

According to the radiation irradiation detection system, the radiation generation apparatus, and the radiation detection apparatus of the present invention, in a case where an emission permitting signal output from the radiation detection apparatus is received, the radiation generation apparatus emits pulsed radiation, and controls emission of the pulsed radiation by using the emission permitting signal received in a period in which the radiation is not emitted during an emission period of the pulsed radiation.

Consequently, since it is possible to reduce the influence of noise caused by emission of radiation on the emission permitting signal, imaging can be prevented from being wrongly stopped as described above, and thus it is possible to reduce a radiation exposure dose of a patient without performing unnecessary reimaging.

### BRIEF DESCRIPTION OF THE DRAWINGS

FIG. 1 is a block diagram illustrating a schematic configuration of a radiation irradiation detection system according to an embodiment of the present invention.

FIG. 2 is a timing chart illustrating a relationship between output of a radiation emission permitting signal and emission of pulsed radiation.

FIG. 3 is a timing chart illustrating a relationship between output of a radiation emission stopping signal and a cycle of pulsed radiation.

FIG. 4 is a diagram for explaining operation control for a radiation image detector.

FIG. 5 is a flowchart for explaining an operation of the radiation irradiation detection system according to the embodiment of the present invention.

### DETAILED DESCRIPTION

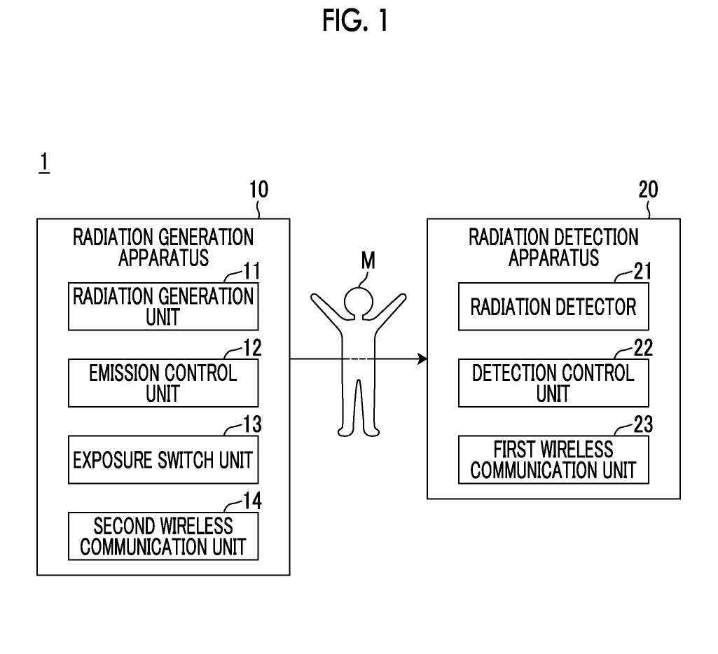

Hereinafter, a radiation irradiation detection system and a radiation generation apparatus according to an embodiment of the present invention will be described in detail with reference to the drawings. FIG. 1 is a block diagram illustrating a schematic configuration of a radiation irradiation detection system of the present embodiment.

A radiation irradiation detection system 1 of the present embodiment includes, as illustrated in FIG. 1, a radiation generation apparatus 10 and a radiation detection apparatus 20. The radiation generation apparatus 10 is preferably portable, and the radiation detection apparatus 20 is also preferably a portable cassette. However, the present invention is not limited to a portable radiation irradiation detection system, and is applicable to an installation type radiation irradiation detection system such as a radiation irradiation detection system imaging a standing subject and a radiation irradiation detection system imaging a lying subject.

The radiation generation apparatus 10 emits radiation toward a subject M such as a patient, and includes a radiation generation unit 11 which generates radiation, an emission control unit 12 which controls emission of radiation, an exposure switch unit 13, and a second wireless communication unit 14.

The radiation generation unit 11 includes a radiation source such as an X-ray tube, and generates radiation by applying a high voltage thereto.

The emission control unit 12 includes, for example, a central processing unit (CPU), and controls emission of radiation from the radiation generation apparatus 10. Specifically, the emission control unit 12 controls a tube voltage and a tube current applied to the radiation generation unit 11, and controls the intensity and an emission time of radiation emitted from the radiation generation unit 11 by controlling the tube voltage or the tube current.

The emission control unit 12 of the present embodiment receives a radiation irradiation instruction via the exposure switch unit 13, and causes radiation to be emitted while an emission permitting signal output from a detection control unit 22 which will be described later of the radiation detection apparatus 20 is being received. The emission permitting signal is a signal for permitting radiation to be emitted. In the present embodiment, the emission permitting signal is output to the radiation generation apparatus 10 from the detection control unit 22 in a preset cycle from a time point at which a preparation operation of a radiation detector 21 of the radiation detection apparatus 20 is completed.

In the present embodiment, the emission permitting signal is transmitted from a first wireless communication unit 23 of the radiation detection apparatus 20 as a wireless signal and is received by the second wireless communication unit 14 of the radiation generation apparatus 10, but the emission permitting signal may not be normally received by the radiation generation apparatus 10 due to, for example, the influence of noise. Particularly, a wireless signal transmitted from the first wireless communication unit 23 is greatly influenced by noise caused by radiation emitted from the radiation generation apparatus 10.

In this case, as described above, in a case where emission of radiation is controlled on the basis of the emission permitting signal output from the radiation detection apparatus 20, the emission permitting signal cannot be normally received, and thus emission of radiation is stopped. In other words, capturing of a radiation image is wrongly stopped although the radiation image can be normally captured. In a case where imaging is stopped as mentioned above, reimaging is required to be performed, and thus there is a problem in that a radiation exposure dose of a patient increases.

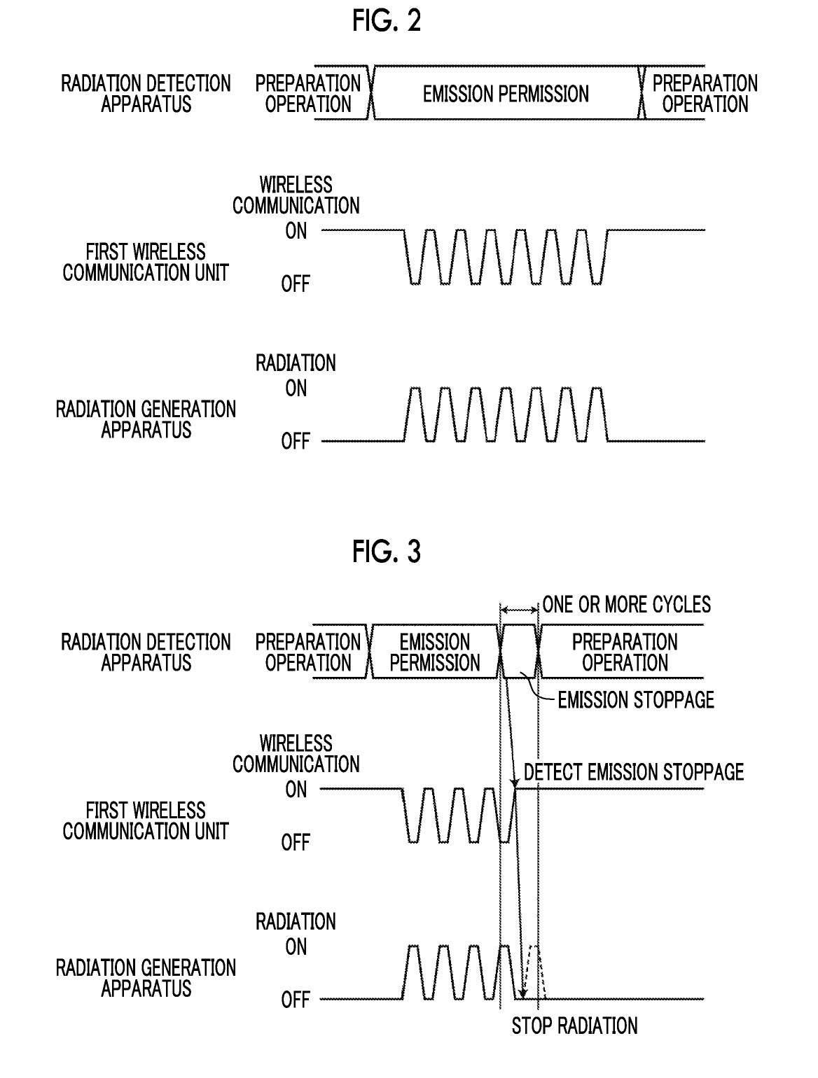

Therefore, the emission control unit 12 of the present embodiment emits pulsed radiation as illustrated in FIG. 2 in a case where the emission permitting signal is received. During an emission period of the pulsed radiation, emission of the pulsed radiation is controlled by using an emission permitting signal received by the second wireless communication unit 14 in a period in which radiation is not emitted. As illustrated in FIG. 2, the first wireless communication unit 23 of the radiation detection apparatus 20 outputs the emission permitting signal in only a period in which radiation is not emitted.

Consequently, it is possible to reduce the influence of noise caused by radiation on the emission permitting signal, and thus the emission control unit 12 can normally receive the emission permitting signal. Therefore, since capturing of a radiation image can be prevented from being stopped, it is not necessary to perform reimaging, and thus it is possible to reduce a radiation exposure dose of a patient.

A cycle of the pulsed radiation is preferably 10 ms or more and 100 ms or less.

A cycle and a duty ratio of the pulsed radiation are preferably changed depending on imaging conditions such as patient information, an imaging part, and an imaging method. For example, a table in which a cycle and/or a duty ratio of the pulsed radiation is correlated with imaging conditions may be set in advance.

In the present embodiment, as illustrated in FIG. 2, the first wireless communication unit 23 outputs the emission permitting signal only in a period in which radiation is not emitted, but this is only an example, and the first wireless communication unit 23 may continuously output emission permitting signals. The emission control unit 12 may control emission of pulsed radiation by using an emission permitting signal received in a period in which radiation is not emitted among emission permitting signals received by the second wireless communication unit 14. Conversely, the emission control unit 12 may not use an emission permitting signal received in a period in which radiation is not emitted among emission permitting signal received by the second wireless communication unit 14 in a case where emission of pulsed radiation is controlled.

The emission control unit 12 of the present invention stops emission of radiation in a case where a radiation emission stopping instruction is received via the exposure switch unit 13, or an emission stopping signal output from the first wireless communication unit 23 of the radiation detection apparatus 20 is received. For example, in a case where abnormality in the radiation detector 21 is detected, the detection control unit 22 of the radiation detection apparatus 20 outputs the emission stopping signal from the first wireless communication unit 23. The first wireless communication unit 23 is controlled by the detection control unit 22, and continuously outputs the emission stopping signal during at least one cycle of pulsed radiation as illustrated in FIG. 3.

Consequently, since the emission control unit 12 can more reliably recognize the emission stopping signal, and can thus more reliably stop emission of radiation, it is possible to prevent an increase in a radiation exposure dose of a patient due to unnecessary emission of radiation.

Referring to FIG. 1, as described above, the second wireless communication unit 14 receives the emission permitting signal and the emission stopping signal transmitted from the first wireless communication unit 23 of the radiation detection apparatus 20.

The exposure switch unit 13 receives a radiation irradiation instruction and a radiation stoppage instruction given by a user. Specifically, in the present embodiment, an irradiation instruction is given by turning on the exposure switch unit 13, and an emission stopping instruction is given by turning off the exposure switch unit 13.

Next, the radiation detection apparatus 20 will be described. The radiation detection apparatus 20 includes the radiation detector 21, the detection control unit 22, and the first wireless communication unit 23.

The radiation detector 21 detects radiation which is output from the radiation generation apparatus 10 and is transmitted through the subject M, and outputs a radiation detection signal. As the radiation detector 21, for example, a radiation detector including a scintillator (phosphor) which converts incident radiation into visible light, and a thin film transistor (TFT) active matrix substrate may be used. The radiation detector 21 is not limited thereto, and a so-called direct conversion type radiation detector which directly converts incident radiation into an electric charge signal may be used.

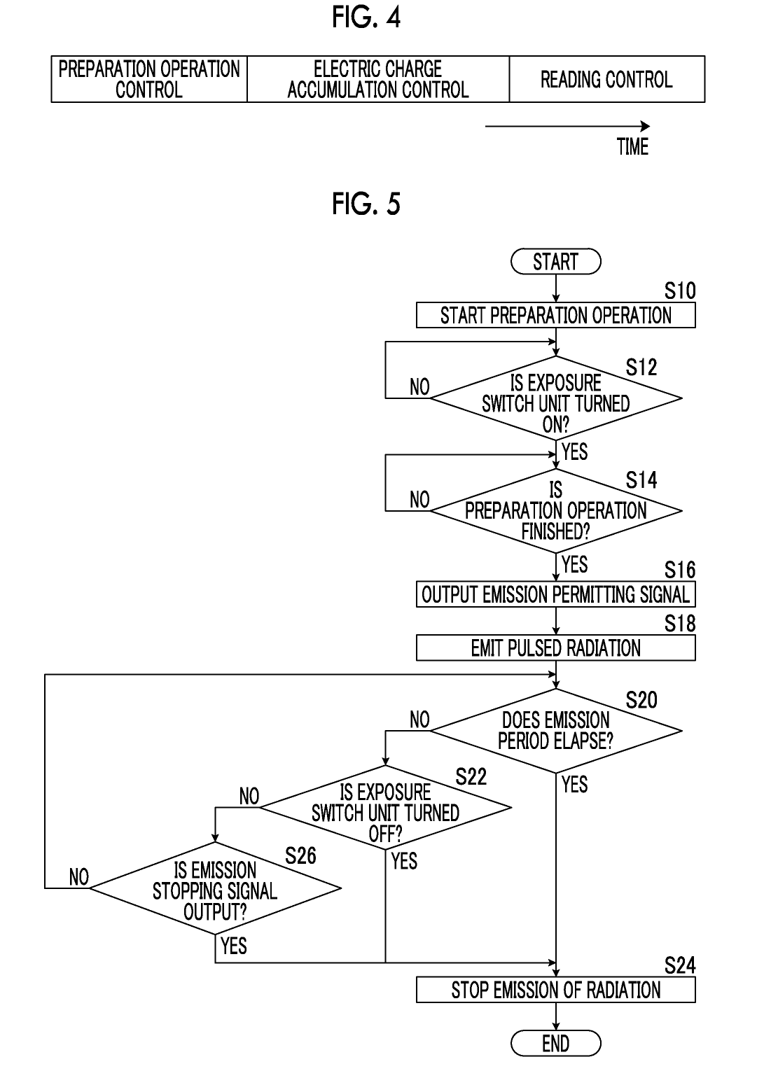

The detection control unit 22 includes, for example, a CPU, and controls an operation of the radiation detector 21. Operation control of the radiation detector 21 includes preparation operation control, electric charge accumulation control, and reading control as illustrated in FIG. 4.

In a period of the preparation operation control, a high voltage is applied to the radiation detector 21, and a preparation operation is performed such that the radiation detector 21 is brought into a state of being capable of detecting radiation.

The detection control unit 22 starts the electric charge accumulation control after the preparation operation is completed. Specifically, the detection control unit 22 controls the radiation detector 21 to start accumulation of electric charge generated by irradiation with radiation transmitted through the subject M. The detection control unit 22 outputs an emission permitting signal to the radiation generation apparatus 10 from a time point at which the electric charge accumulation operation is started. The emission control unit 12 causes radiation to be emitted in a case where an irradiation starting instruction is received by turning on the exposure switch unit 13, and the emission permitting signal is received. Radiation transmitted through the subject M is detected by the radiation detector 21. Emission of radiation is performed in only a preset emission period as long as an emission stopping instruction is not received via the exposure switch unit 13.

The detection control unit 22 starts the reading control from a time point at which the radiation emission period is finished. Specifically, the detection control unit 22 controls the radiation detector 21 to start reading of the electric charge signals accumulated in the radiation emission period. Radiation detection signals corresponding to the electric charge signals read from the radiation detector 21 are stored in a storage medium such as a memory provided in the radiation detection apparatus 20. The radiation detection signals stored in the storage medium undergo predetermined signal processing, and are output to an apparatus such as a console.

The first wireless communication unit 23 transmits a radiation emission permitting signal and a radiation emission stopping signal to the radiation generation apparatus 10 as described above. In the present embodiment, as described above, the first wireless communication unit 23 outputs an emission permitting signal in a period in which radiation is not emitted during an emission period of pulsed radiation. The first wireless communication unit 23 continuously outputs an emission stopping signal during at least one cycle of the pulsed radiation.

In the present embodiment, for example, in a case where abnormality in the radiation detector 21 is detected, the emission stopping signal is output from the first wireless communication unit 23, and thus emission of radiation is stopped, but the present embodiment is not limited thereto, and emission of radiation may be stopped by stopping output of an emission permitting signal from the first wireless communication unit 23. In this case, the first wireless communication unit 23 preferably stops outputting of an emission permitting signal during at least one cycle of pulsed radiation.

Consequently, since the emission control unit 12 can more reliably recognize stoppage of outputting of an emission permitting signal, and can thus more reliably stop emission of radiation, it is possible to prevent an increase in a radiation exposure dose of a patient due to unnecessary emission of radiation.

Next, a description will be made of an operation of the radiation irradiation detection system of the present embodiment with reference to a flowchart of FIG. 5.

First, in the radiation generation apparatus 10, a preparation operation of the radiation generation unit 11 is started by the emission control unit 12, and, in the radiation detection apparatus 20, a preparation operation of the radiation detector 21 is started under the control of the detection control unit 22 (S10).

In a case where the subject M is located at a position with respect to the radiation detector 21, and then the exposure switch unit 13 is turned on by a radiologist or the like (S12, YES), it is checked whether or not the preparation operations of the radiation generation unit 11 and the radiation detector 21 are finished (S14).

In a case where it is checked that the preparation operations of the radiation generation unit 11 and the radiation detector 21 are finished (S14, YES), the detection control unit 22 of the radiation detection apparatus 20 starts to output an emission permitting signal to the radiation generation apparatus 10 (S16), and starts an electric charge accumulation operation in the radiation detector 21.

In a case where the emission permitting signal is received in the radiation generation apparatus 10, the emission control unit 12 starts to emit pulsed radiation (S18). After the pulsed radiation starts to be emitted, the detection control unit 22 outputs an emission permitting signal in a period in which radiation is not emitted as described above, and the emission permitting signal is received by the radiation generation apparatus 10.

Next, it is checked whether or not the exposure switch unit 13 is turned off in the preset radiation emission period (S20, NO), and, in a case where the exposure switch unit 13 is still turned on (S24, NO), it is checked whether or not an emission stopping signal is output from the radiation detection apparatus 20. In a case where an emission stopping signal is not output (S26, NO), the emission control unit 12 causes radiation to be continuously emitted.

In a case where the preset radiation emission period elapses (S20, YES), the emission control unit 12 stops emission of radiation (S24). The detection control unit 22 finishes the electric charge accumulation operation in the radiation detector 21, and performs a reading operation.

On the other hand, in a case where the exposure switch unit 13 is turned off (S24, YES) or an emission stopping signal is output from the radiation detection apparatus 20 (S26, YES) in the preset radiation emission period (S20, NO), the emission control unit 12 stops emission of radiation (S22).

In a case where radiation imaging is stopped by turning off the exposure switch unit 13, or radiation imaging is stopped due to an emission stopping signal being output from the radiation detection apparatus 20, the detection control unit 22 may stop an operation of the radiation detector 21, and may finish an electric charge accumulation operation in the radiation detector 21 so as to perform a reading operation as usual. As mentioned above, an operation as usual is performed, and a radiation image is acquired, and thus it is possible to determine whether or not reimaging is required to be performed.

What is claimed is:

1. A radiation irradiation detection system comprising:
a radiation generation apparatus that includes
a radiation generation unit generating radiation, and
an emission control unit controlling emission of the radiation; and
a radiation detection apparatus that includes
a radiation detector detecting the radiation transmitted through a subject,
wherein the radiation detection apparatus further includes a first wireless communication unit that outputs an emission permitting signal for permitting the radiation to be emitted as a wireless signal,
wherein the radiation generation apparatus further includes a second wireless communication unit that receives the emission permitting signal, and
wherein, in a case where the emission permitting signal is received, the emission control unit causes pulsed radiation to be emitted, and controls emission of the pulsed radiation by using the emission permitting signal received by the second wireless communication unit in a period in which the radiation is not emitted during an emission period of the pulsed radiation.

2. The radiation irradiation detection system according to claim 1,
wherein the first wireless communication unit outputs the emission permitting signal in only the period in which the radiation is not emitted.

3. The radiation irradiation detection system according to claim 1,
wherein the first wireless communication unit continuously outputs the emission permitting signal, and
wherein the emission control unit controls emission of the pulsed radiation by using the emission permitting signal received in the period in which the radiation is not emitted among the emission permitting signals received by the second wireless communication unit.

4. The radiation irradiation detection system according to claim 1,
wherein, in a case of stopping emission of the radiation, the first wireless communication unit outputs an emission stopping signal for instructing emission of the radiation to be stopped as a wireless signal, and continuously outputs the emission stopping signal during at least one cycle of the pulsed radiation.

5. The radiation irradiation detection system according to claim 1,
wherein, in a case of stopping emission of the radiation, the first wireless communication unit stops outputting of the emission permitting signal during at least one cycle of the pulsed radiation.

6. The radiation irradiation detection system according to claim 1
wherein, in a case where a preparation operation of the radiation detector is finished, the radiation detection apparatus outputs the emission permitting signal, and starts an operation of accumulating a detection signal of the radiation in the radiation detector.

7. The radiation irradiation detection system according to claim 1,
wherein the radiation generation apparatus is portable.

8. A radiation generation apparatus comprising:
a radiation generation unit that generates radiation;
an emission control unit that controls emission of the radiation; and
a wireless communication unit that performs wireless communication,
wherein, in a case where an emission permitting signal as a wireless signal output from a radiation detection apparatus detecting the radiation transmitted through a subject is received by the wireless communication unit, the emission control unit causes the pulsed radiation to be emitted, and controls emission of the pulsed radiation by using the emission permitting signal received in a period in which the radiation is not emitted during an emission period of the pulsed radiation.

9. The radiation generation apparatus according to claim 8,
wherein the radiation generation apparatus is portable.

10. A radiation detection apparatus comprising:
a radiation detector that detects radiation transmitted through a subject; and
a wireless communication unit that performs wireless communication,
wherein the wireless communication unit outputs an emission permitting signal for permitting the radiation to be emitted as a wireless signal to a radiation generation apparatus, and outputs the emission permitting signal in only a period in which the radiation is not emitted during an emission period of pulsed radiation emitted from the radiation generation apparatus in response to the emission permitting signal.
